(19) United States
(12) Patent Application Publication
(10) Pub. No.: US 2006/0178520 A1
(43) Pub. Date: Aug. 10, 2006

(54) Process for preparing medrogestone

(75) Inventors: Jean-Luc Moutou, Cagnes sur Mer (FR); Benoit Rondot, La Colle sur Loup (FR); Jean Lafay, Nice (FR); Philippe Maillos, Hannover (FR)

Correspondence Address: CROWELL & MORING LLP;INTELLECTUAL PROPERTY GROUP, P.O. BOX 14300, WASHINGTON, DC, 20044-4300, US

(73) Assignee: Solvay Pharmaceuticals GmbH, Hannover (DE)

(21) Appl. No.: 11/332,297

(22) Filed: Jan. 17, 2006

Related U.S. Application Data
(60) Provisional application No. 60/644,053, filed on Jan. 18, 2005.

(51) Int. Cl. C07J 7/00

(52) U.S. Cl. 552/607

(57) ABSTRACT

A method for preparing medrogestone by heterogeneously palladium-catalyzed isomerization from 17α-methyl-6-methylenepregn-4-ene-3,20-dione.

### CROSS REFERENCE TO RELATED APPLICATION

This application claims priority from U.S. provisional patent application No. 60/644,053, filed Jan. 18, 2005, the entire disclosure of which is incorporated herein by reference.

### BACKGROUND OF THE INVENTION

The present invention relates to a method for preparing medrogestone by heterogeneously palladium-catalyzed isomerization from 17α-methyl-6-methylenepregn-4-ene-3,20-dione.

Medrogestone is a 6-methyl steroid which is used e.g. for the treatment of certain disorders in women's menstrual flow or to supplement estrogen treatment in the climacteric period.

U.S. Pat. No. 3,170,936 describes a method for preparing medrogestone by acid-catalyzed elimination of water from 5α,6β-dihydroxy-6α-17α-dimethylpregnan-3,20-dione.

A method for preparing, inter alia, medrogestone is known from European Patent No. EP 085,900, in which a corresponding Δ⁴-3-ketosteroid is reacted with e.g. methoxymethyl acetate in the presence of catalytic amounts of phosphorus oxychloride and an alkali metal acetate.

European Patent No. 680,970 discloses a method for preparing medrogestone from 6β-hydroxy-6α,17α-dimethylpregn-4-ene-3,20-dione by reaction with boron trifluoride etherate. 17α-methyl-6-methylenepregn-4-ene-3,20-dione produced as byproduct is regarded as disruptive in this known method and has to be removed by reaction with maleic anhydride in order to obtain pure medrogestone.

Methods for isomerization of a 6-methylene-4-en-3-one system on different steroid compounds for preparing the biologically valuable 6-methyl-4,6-dienone system are known for example from D. Burn et al., Tetrahedron 21 (1965) 1619-1624 or from F. Schneider et al., Helv. Chim. Acta 56/7 (1973) 2396-2404, but in the methods given therein the addition of a hydrogen donor is deemed obligatory when performing the heterogeneously catalyzed isomerization reaction. Further isomerization reactions are known, for example, from Published German Patent Application No. DE 25 22 533 (=GB 1,515,284) and from U.S. Pat. No. 4,544,555. Furthermore, the steroid systems listed above as examples contain no methyl group in position 17 of the steroid structure. It is assumed that substituents in position 17 of the steroid structure considerably reduce the reactivity of a steroid derivative for an isomerization reaction by steric screening (cf. e.g. L. F. Fieser and M. Fieser “Steroide”, Verlag Chemie, Weinheim/Bergstr. 1961, pp. 10-20), in particular if at the same time a further substituent such as a methyl group is located in position 18 of the steroid structure.

### SUMMARY OF THE INVENTION

It was an object of the invention to provide a novel method for preparing medrogestone which is simple and inexpensive to carry out and accordingly is also suitable for large-scale industrial synthesis.

It has now been found that medrogestone can be obtained simply and inexpensively by heterogeneously catalyzed isomerization from 17α-methyl-6-methylenepregn-4-ene-3,20-dione and purification and processing steps which can be carried out subsequently if desired. The addition of a hydrogen donor such as elemental hydrogen or cyclohexene when performing the heterogeneously catalyzed isomerization reaction is not necessary according to the method in accordance with the present invention.

The invention therefore relates to a method for preparing medrogestone corresponding to Formula I, 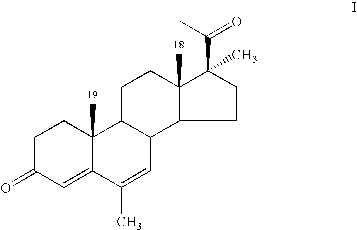
in which 17α-methyl-6-methylenepregn-4-ene-3,20-dione of Formula II 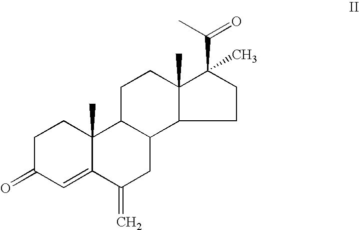
is isomerized in a C₁₋₄ alcohol or in a mixture of such C₁₋₄ alcohols and in the presence of a supported palladium catalyst at a temperature of 60° C. to 95° C. to form a compound of Formula I.

The method is suitable for preparing medrogestone in large-scale industrial synthesis. For example, approximately 5 to 100 kg, preferably 25 to 60 kg, of the starting compound of Formula II can be used. The yield in that case, depending on, inter alia, the number of purification stages, is up to 95%, relative to the mass of starting compound of Formula II used.

The C₁₋₄ alcohols suitable as solvents or components of solvent mixtures may be straight-chain or branched. Preferred solvents are ethanol, methanol, 2-propanol and mixtures of these aforementioned alcohols. Ethanol, especially absolute ethanol, is particularly preferred. The solvent or solvent mixture is usually calculated in a ratio of 8 to 15 volume units, relative to one mass unit of starting compound of Formula II. A volume unit (=VU) in the context of the present invention is calculated in liters. A mass unit (=MU) in the context of the present invention is calculated in kilograms.

In particular palladium on aluminum oxide (=Pd/Al₂O₃), palladium on activated carbon (=Pd/C) and/or palladium on calcium carbonate can be used as supported palladium catalysts. Usually 5% Pd/C or 5% Pd/Al₂O₃ is used. 5% Pd/C is preferred. The supported palladium catalyst is usually calculated in a ratio of from 0.15 to 0.25 MU, relative to one MU of starting compound of Formula II.

The reaction is carried out at a temperature of 60° C. to 95° C., usually at the boiling temperature of the solvent or solvent mixture. Preferably the reaction temperature is greater than or equal to 72° C., in order to avoid the formation of byproducts as far as possible. In a particularly preferred variant, the reaction is carried out at the boiling temperature of ethanol, in particular denatured ethanol.

Usually the supported palladium catalyst and the solvent are placed in a suitable reaction vessel such as a suitably sized stainless steel container under a protective gas atmosphere and with moisture excluded and heated under reflux cooling to a temperature of 60° C. to 95° C., in particular to boiling, before the starting compound of Formula II is added to this initial solution. The reaction is then continued under reflux cooling at boiling heat, until as complete a reaction as possible has occurred. The progress of the reaction can be followed in known manner, for example, by high-performance liquid chromatography (=HPLC) or by thin-layer chromatography (=TLC). Typical reaction times are from 1.5 to 3 hours. Then the resulting medrogestone can be isolated and/or purified if desired.

To isolate medrogestone, in a first variant the reaction mixture can be cooled to approx. 35 to 50° C. and be filtered through a known filter. Then the solvent can be largely evaporated, for example to approximately one third of the starting volume, from the filtrate at reduced pressure in known manner. The remaining residue can then be cooled for approximately 1 to 2 hours to temperatures of 0° C. to 5° C., with medrogestone precipitating as a solid, preferably in crystalline form. The resulting solid medrogestone can then also be dried in known manner, for example at reduced pressure and a temperature of 40° C. for a duration of approximately 6 to 10 hours.

To isolate medrogestone, in a second variant the reaction mixture can be filtered at a temperature of 65° C. to 75° C. in known manner through a suitable filter and the volume of the filtrate can then be evaporated at reduced pressure in known manner to approximately 5.5 to 6.5 VU, relative to one MU of starting compound of Formula II. The resulting filtrate which has been reduced in volume can then be heated under reflux cooling to a temperature above room temperature, in particular to boiling, before from 3.5 to 4.5 VU water, relative to one MU of starting compound of Formula II, are added at this elevated temperature. Once the water has been added, the aqueous alcohol solution can then be cooled to a temperature of 0° C. to 15° C. It is particularly advantageous to cool the reaction solution in steps, in particular to remain at the elevated starting temperature for from 5 to 10 minutes after the addition of the water, then to cool it to 18 to 24° C. for from 25 to 35 minutes. and finally to cool it for from 50 to 70 minutes to 0° C. to 15° C., in particular to 10° C., with medrogestone precipitating as solid, preferably in crystalline form. The resulting solid medrogestone can then if desired also be dried in known manner, for example at reduced pressure and a temperature of 20° C. to 40° C. for a duration of approximately 6 to 36 hours.

For purification of medrogestone, in a first variant the solid medrogestone obtained for isolation according to the first or second variant can be dissolved in a suitable inert polar organic solvent or a solvent mixture at a temperature above room temperature, in particular at the boiling temperature of the solvent or solvent mixture, and then can be precipitated again by cooling to room temperature or to a temperature below room temperature. In this variant methanol, ethanol, 2-propanol and mixtures thereof can be used as solvent. In particular ethanol can be used, preferably from 3.5 to 4.5 volume units of ethanol, relative to one MU of solid medrogestone obtained according to the first or second variant. For example, one MU of medrogestone can be added to from 3.5 to 4.5 VU of ethanol and be heated to boiling for from 5 to 15 minutes under reflux cooling. Then the batch can be cooled to 5° C. to 15° C. and be kept at this temperature for from 25 to 35 minutes before being cooled to 5° C. and maintained for from 45 to 90 minutes at this temperature. The resulting crystals of medrogestone can then be filtered out and dried in known manner.

For purification of medrogestone, in a second variant the solid medrogestone obtained for isolation according to the first or second variant or the crystals of medrogestone obtained for purification according to the first variant can be dissolved in a suitable inert polar organic solvent or a solvent mixture at a temperature above room temperature, in particular at the boiling temperature of the solvent or solvent mixture, and then be precipitated again by cooling to room temperature or to a temperature below room temperature. In this variant in particular 2-propanol can be used as solvent, preferably from 3.5 to 4.5 VU 2-propanol, relative to one MU of solid medrogestone obtained according to the first or second variant or crystals of medrogestone obtained for purification according to the first variant. In particular, one MU of medrogestone can be added to 3.5 to 4.5 VU of 2-propanol and heated to boiling under reflux cooling. Once the medrogestone has completely dissolved, the batch is filtered in known manner and the resulting filtrate is cooled, preferably at a rate of 2° C./minute. It is advantageous during the cooling operation to seed the batch with small amounts of crystalline medrogestone. The resulting crystals of medrogestone can then be washed with 2-propanol (preferably with 0.4 to 0.6 VU, relative to one MU of medrogestone solid used) and filtered out and dried to a desired specification in a known manner.

The solid medrogestone obtained according to one of the isolation and/or purification methods described above can then also be micronized in a known manner.

The compound of Formula II can be obtained by initially reacting a compound corresponding to Formula III,

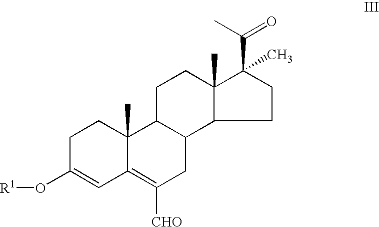

wherein R¹ represents C₁₋₄ alkyl, with an alkali metal hydride in a polar organic solvent which is inert under the reaction conditions or in a solvent mixture and then reacting the resulting intermediate with a reagent suitable for cleaving the C₁₋₄ alkyl ether. In the context of the present compounds, C₁₋₄ alkyl groups may be branched or unbranched. The preferred C₁₋₄ alkyl group is ethyl.

Suitable solvents or solvent mixtures include, for example, methanol; ethanol; 1-propanol; 2-propanol; tert butanol; 2-butanol; 1-butanol; tetrahydrofuran (THF); 1,4-dioxane; acetone and also mixtures of two or more of the aforementioned solvents. Ethanol, tetrahydrofuran, acetone and mixtures thereof are preferred. The solvent or solvent mixture is usually calculated in a ratio of 6 to 10 VU, relative to one MU of the starting compound of Formula III.

Suitable alkali metal hydrides include, for example, LiAlH₄, NaBH₄, NaBH₃CN or Zn(BH₄)₂. NaBH₄ is preferred.

Suitable reaction temperatures are from −10° C. to 0° C., preferably −5° C.

Usually one MU of starting compound of Formula III, preferably 17□-methyl-3-ethoxy-6-formylpregna-3,5-dien-20-one, is dissolved or suspended in from 3.5 to 4.5 VU solvent or solvent mixture, in particular ethanol, relative to one MU of starting compound of Formula III, and it is cooled to an internal temperature of the reaction mixture of 5° C. to 15° C. From 0.02 to 0.04 MU of alkali metal hydride, preferably NaBH₄, are added to this initial solution, followed by 0.04 to 0.06 VU acetone, and 3.5 to 4.5 VU tetrahydrofuran, each relative to one MU of starting compound of Formula III. The reaction mixture then is cooled further to an internal temperature of from −10° C. to 0° C., preferably −5° C. Then the C₁₋₄ alkyl ether can be acid-cleaved, preferably in a one-pot reaction, for example by addition of a dilute aqueous sulfuric acid solution (usually 0.1 VU sulfuric acid and 0.1 VU water, each relative to one MU of starting compound of Formula III). Once the reaction has taken place, the reaction mixture can be neutralized with an alkali metal carbonate, such as potassium carbonate, or an aqueous solution of the alkali metal carbonate. The resulting product of Formula II can then be precipitated by addition of water to the reaction mixture and may be isolated and purified in a known manner.

Compounds of Formula III can be obtained by reacting a compound of Formula IV,

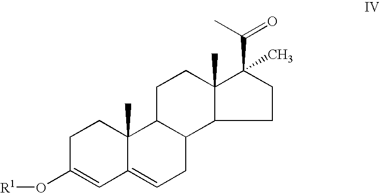

wherein R¹ has the above meaning, in an organic solvent which is inert under the reaction conditions or in a solvent mixture with an N-disubstituted formamide, preferably DMF, and phosphorus oxychloride.

The reaction can be carried out under the known conditions of what is called a Vilsmeier formylation, the starting compound of Formula IV being reacted with from 0.20 to 0.35 VU, preferably 0.28 MU, of dimethylformamide, relative to one MU of starting compound of Formula IV, and 0.45-0.50 MU, preferably 0.473 MU, phosphorus oxychloride, relative to one MU of starting compound of Formula IV.

Suitable solvents or solvent mixtures also include open-chain or cyclic di-lower alkyl ethers such as diethyl ether; diisopropyl ether; tetrahydrofuran; 1,4-dioxane, and also mixtures of the aforementioned solvents. Dimethylformamide can also be used as the solvent. Tetrahydrofuran is preferred as the solvent. The solvent or solvent mixture is usually calculated in a ratio of from 1.5 to 6 VU, preferably 1.5 to 2.5 VU, relative to one MU of the starting compound of Formula IV.

Suitable reaction temperatures are −25° C. to −5° C., preferably approximately −15° C.

It is advantageous to carry out the reaction in the presence of at least catalytic amounts of a non-nucleophilic organic base such as a nitrogen base, for example triethylamine.

Once the reaction is completed, an amount, sufficient for neutralisation, of a base such as an alkali metal carbonate, for example potassium carbonate, or an aqueous solution of an alkali carbonate is added to the reaction mixture with cooling to −10° C. to 10° C. For example, a solution of from 0.8 to 0.9 MU, preferably 0.853 MU, of potassium carbonate, in from 1.5 to 2 VU, preferably in 2 VU, of water, in each case relative to one MU of starting compound of Formula IV, can be added.

The resulting compound of Formula III can then also be purified and/or isolated. To this end, the batch, after thawing to 15° C. to 25° C., can initially be diluted with from 2.5 to 3.5 VU ethanol, relative to one MU of starting compound of Formula IV, and stirred for from 20 to 40 minutes. Then the batch can be diluted with from 9 to 11 VU water, relative to one MU of starting compound of Formula IV, and stirred for a further 20 to 90 min. The resulting precipitate can then be filtered out, subsequently washed with from 1.5 to 2.5 VU water, relative to one MU of starting compound of Formula IV, and partially dried, for example in a vacuum for from 1 to 8 hours. Then the initially dried precipitate can be stirred with from 4 to 6 VU water, relative to one MU of starting compound of Formula IV, and at least a catalytic amount of a non-nucleophilic organic base, such as a nitrogen base, preferably with from 0.04 to 0.06 VU triethylamine, relative to one MU of starting compound of Formula IV, at a temperature of 15° C. to 30° C. for from 30 to 120 minutes. Then the precipitate can be filtered out again and washed with water until no chloride ions are indicated in the filtrate. For purification, the resulting precipitated compound of Formula III can be heated to a temperature above room temperature, preferably under reflux cooling and to boiling temperature of the solvent, with from 1.5 to 2.5 VU ethanol, relative to one MU of starting compound of Formula IV, and at least a catalytic amount of a non-nucleophilic organic base such as a nitrogen base, preferably with from 0.01 to 0.03 VU triethylamine, relative to one MU of starting compound of Formula IV, until the compound of Formula III dissolves. After cooling to room temperature, the resulting recrystallized compound of Formula III can be washed with from 0.4 to 0.6 VU ethanol, relative to one MU of starting compound of Formula IV, and dried to a desired specification. Yields of 68-85% of the compound of Formula III are obtained, relative to the starting compound of Formula IV which is used.

Compounds corresponding to formula IV can be prepared by alkylating an enol ether corresponding to formula V,

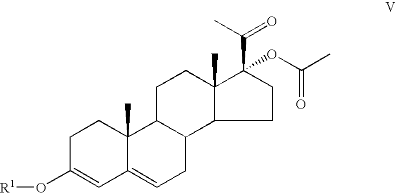

wherein R¹ has the above meaning, in known manner under the conditions of what is called a Birch reduction with lithium in liquid ammonia with a methyl halide, preferably methyl iodide.

Compounds of Formula V are known per se, for example from U.S. Pat. No. 3,240,671, and can be prepared according to the methods described in that patent, analogous methods, or other methods. For example, compounds of Formula V can be prepared by reacting known 17□-acetoxyprogesterone in known manner with a suitable orthoester.

### EXAMPLES

### Example 1

### 6,17α-dimethylpregna-4,6-diene-3,20-dione (medrogestone, compound of Formula I)

A) 100 kg of 17α-acetoxyprogesterone (VI) are dissolved in 400 liters of absolute ethanol. 100 liters of triethyl orthoformate and 500 grams of p-toluenesulfonic acid are added thereto. The mixture is heated for 4 hours to 35° C., allowed to cool to room temperature (=RT). 7.26 grams of triethylamine are added, and the mixture is cooled to 0° C. for 1 hour. The resulting solid is filtered out and washed at 0° C. with 50 liters of cold ethanol containing 1 vol. % triethylamine. The washed solid is left under reflux cooling and at boiling heat in 800 liters of diisopropyl ether containing 1 vol. % triethylamine until the solid is dissolved. Then approx. 200 liters of diisopropyl ether is distilled off, the solution is cooled to 0° C. for 1 hour, and the 17α-acetoxy-3-ethoxy-3,5-pregnadien-20-one (V) occurring as solid is filtered out and dried at temperatures below 40° C. and at reduced pressure for 6 hours.

B) 3.58 kg of lithium are added at −70° C. to 640 liters of liquid ammonia over 15 minutes. After 30 minutes, a solution of 80 kg of an ethyl enol ether as obtained above in 1280 liters of tetrahydrofuran is added for 2 hours, with the internal temperature being kept at approximately −60° C. to −65° C. Once addition is complete, the reaction mixture is kept for 1 hour at −65° C., then 74.4 liters of methyl iodide are added over 15 minutes. The temperature is maintained at −65° C. for a further 2 hours, and then the ammonia is evaporated off. Tetrahydrofuran is distilled off, and the remaining residue is taken up with 320 liters of toluene. Undissolved solids are filtered out, and the mixture is subsequently washed with 100 liters of toluene. 160 liters of ethyl acetate are added to the filtrate, and the organic phase is washed twice with water. The solvent is evaporated, and a solid is obtained which is recrystallised from 400 liters of methanol containing 1 vol. % of triethylamine. Cooling of the mother liquor for 1 hour to 0° C. yields 51.3 kg of 17α-methyl-3-ethoxy-3,5-pregnadien-20-one (IV) as a white crystalline powder having a melting point of 114 to 116° C.

C) 70 kg of a 17α-methyl-3-ethoxy-3,5-pregnadien-20-one as obtained above are suspended in 140 liters of tetrahydrofuran in an enameled reaction vessel, and 700 grams of triethylamine and 19.6 liters of dimethylformamide are added. This initial solution is cooled to −15° C. before 33.1 kg of phosphorus oxychloride are added. The reaction is continued until complete reaction has taken place as determined by HPLC monitoring, then a cooled solution of 59.7 kg of potassium carbonate in 140 liters of purified water is added, stirred for 1 hour and allowed to thaw to 20° C. 210 liters of denatured ethanol is added; the mixture is stirred for 30 minutes; 700 liters of purified water are added, and the mixture is stirred for a further 60 minutes. The resulting solid is vacuum-filtered, subsequently washed with a total of 140 liters of purified water, and dried on the filter for 8 hours in a vacuum. Then the dried solid is taken up with 350 liters of purified water; 3.5 liters of triethylamine are added, and the mixture is stirred for 1 hour at room temperature. Filtration takes place under a vacuum, the solid residue is washed with purified water until no chloride ions can be detected, and then dried for 8 hours in a vacuum at 40° C. Finally the solid is taken up in 140 liters of denatured ethanol, 1.4 liters of triethylamine are added thereto, and the mixture is heated to boiling under reflux cooling until dissolution of the solid is observed. After cooling to room temperature, the 17α-methyl-6-formyl-3-ethoxy-3,5-pregnadien-20-one (III) which again results is filtered out from the mother liquor, subsequently washed with 35 liters of purified water, and then dried for 8 hours in a vacuum at 40° C. The yield is 87.5%, relative to the 17α-methyl-3-ethoxy-3,5-pregnadien-20-one used.

D) 60 kg of a 7α-methyl-6-formyl-3-ethoxy-3,5-pregnadien-20-one as obtained above are suspended in 240 liters of denatured ethanol in a stainless steel reaction vessel and cooled until the internal temperature of the suspension is 10° C. 1.8 kg of NaBH₄ are added to this initial solution, and stirring is continued until complete reaction has occurred as indicated by HPLC monitoring. 3 liters of acetone and 240 liters of tetrahydrofuran are added, and the batch is cooled to −5° C. Then a solution of 6 liters of sulfuric acid in 6 liters of purified water is added and is stirred for 40 minutes, before a solution of 30 kg of potassium carbonate in 66 liters of purified water is added. Stirring is continued until a pH of 7 is achieved; then 900 liters of purified water are added; the resulting suspension is stirred, and the resulting precipitate is filtered out under vacuum and partially dried. The initially dried precipitate is taken up in 240 liters of purified water, stirred at room temperature and filtered in a vacuum. The solid is washed with purified water until no sulfate ions are detected, and the pH value of the washing water is neutral. The washed solid is then dried for 8 hours in a vacuum at 40° C., is then taken up in 200 liters of denatured ethanol and heated under reflux cooling until the solid is dissolved as completely as possible. Then the solution is cooled to room temperature; and the resulting precipitate is filtered out, subsequently washed with 20 liters of denatured ethanol and dried in a vacuum. The initially dried precipitate is taken up in 76 liters of tetrahydrofuran and heated under reflux cooling and with stirring until the solid is dissolved as completely as possible. Then the solution is cooled to room temperature, the resulting precipitate is filtered out and subsequently washed with 19 liters of tetrahydrofuran. Then the resulting 17α-methyl-6-methylenepregn-4-ene-3,20-dione (II) is dried in a vacuum for 8 hours at 40° C. The yield is 62.7%, relative to the α-methyl-6-formyl-3-ethoxy-3,5-pregnadien-20-one used.

E) 6 kg of 5% Pd/C catalyst are suspended in 297 liters of absolute ethanol in a stainless steel reaction vessel and heated to boiling with stirring and under reflux cooling. 30 kg of a 17α-methyl-6-methylenepregn-4-ene-3,20-dione as obtained above are added to this initial solution, and stirring is continued until complete reaction has occurred as determined by HPLC monitoring. Then the catalyst is filtered out, the solution is subsequently washed with 120 liters of absolute ethanol, and the organic phase is evaporated to a volume of approximately 180 liters. 120 liters of purified water are added to the concentrated solution, and the mixture is cooled to room temperature. The resulting crystals are filtered out under a nitrogen atmosphere, washed in succession with 9 liters of absolute ethanol and 6 liters of purified water and then dried for 8 hours in a vacuum at 40° C. The dried precipitate is taken up in 100 liters of absolute ethanol and heated to boiling with stirring and under reflux cooling until the solid is dissolved as completely as possible. Then the solution is cooled to room temperature; the resulting crystals are vacuum-filtered under a nitrogen atmosphere and subsequently washed with 12.5 liters of absolute ethanol, and the resulting medrogestone (I) is dried in a vacuum at 40° C. The yield is 80.5%, relative to the 17α-methyl-6-methylenepregn-4-ene-3,20-dione used.

F) 25 kg of medrogestone as obtained above are suspended in 100 liters of 2-propanol in a stainless steel reaction vessel and heated to boiling with stirring and under reflux cooling until the solid is dissolved as completely as possible. The resulting solution is filtered and cooled to room temperature at a rate of 2° C./minute, with the mother liquor being initially seeded with medrogestone seed crystals. The resulting crystals of recrystallised medrogestone are vacuum-filtered under a nitrogen atmosphere and subsequently washed with 12.5 liters of 2-propanol. Then the medrogestone crystals are dried at 40° C. in a vacuum. The yield is 88.0%, relative to the medrogestone used before recrystallisation. The resulting dried, recrystallised medrogestone crystals are then micronized in known manner to a defined particle size under a nitrogen atmosphere. The micronised medrogestone is then packaged in polyethylene bags.

### Example 2

### 6,17α-dimethylpregna-4,6-diene-3,20-dione (medrogestone, compound of Formula I)—alternative synthesis

5% Pd/Al₂O₃ (29.25 grams) was heated to boiling in absolute ethanol (1.17 liters) which contained 1% (v/v) purified water, under reflux cooling. 117 grams of 17α-methyl-6-methylenepregn-4-ene-3,20-dione in powder form, prepared in accordance with Example 1D, was quickly added to this initial solution. During the addition the temperature was kept at 75° C. The reflux cooling was maintained for 30 minutes, then the heterogeneous catalyst was filtered out, subsequently washed with hot ethanol (470 ml) and the filtrate was concentrated to approx. 800 ml at reduced pressure. Then water was added until crystalline medrogestone precipitated, which was filtered out and subsequently dried under a nitrogen atmosphere. 108.8 grams of crystalline medrogestone were obtained.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A method for preparing medrogestone corresponding to Formula I: 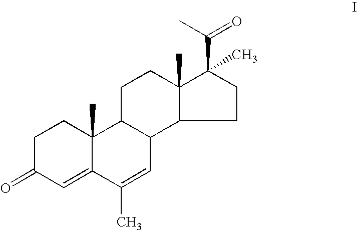
said method comprising isomerizing a compound corresponding to Formula II: 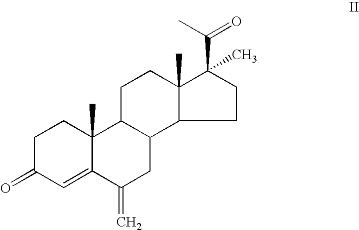
in a C₁₋₄ alcohol or in a mixture of C₁₋₄ alcohols and in the presence of a supported palladium catalyst at a temperature of 60° C. to 95° C. to form the compound of Formula I.

2. A method according to claim 1, wherein the isomerization is carried out in methanol, ethanol, 2-propanol or in a mixture two or more of the aforementioned alcohols.

3. A method according to claim 1, wherein the isomerization is carried out in ethanol.

4. A method according to claim 1, wherein the supported palladium catalyst comprises palladium on aluminium oxide, palladium on activated carbon, or palladium on calcium carbonate.

5. A method according to claim 4, wherein the supported palladium catalyst comprises 5% palladium on activated carbon.

6. A method according to claim 1, wherein the isomerization is carried out at a temperature of at least 72° C.

7. A method according to claim 1, wherein the isomerization is carried out in the absence of an added hydrogen donor.

8. A method according to claim 1, further comprising precipitating the resulting compound of Formula I as a solid.

9. A method according to claim 8, wherein water is added and the solid is precipitated in crystalline form.

10. A method according to claim 8, further comprising dissolving the precipitated solid by addition of from 3.5 to 4.5 volume units of ethanol per mass unit of precipitated solid at a temperature above room temperature, and then re-precipitating the compound of Formula I by cooling the solution to room temperature or a temperature below room temperature.

11. A method according to claim 8, further comprising recrystallizing the precipitated compound of Formula I from 2-propanol.

12. A method according to claim 8, further comprising micronizing the resulting compound of Formula I.

13. A method according to claim 1, wherein the compound of Formula II is obtained by reducing a compound of Formula III, 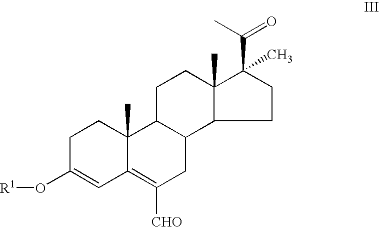
wherein R¹ represents C₁₋₄ alkyl, with an alkali metal hydride in a polar organic solvent or solvent mixture which is inert under the reaction conditions to obtain an intermediate C₁₋₄ alkyl ether, and then reacting the intermediate ether with a reagent suitable for cleaving the ether.

14. A method according to claim 13, wherein the compound of Formula III is obtained by reacting a compound of Formula IV, 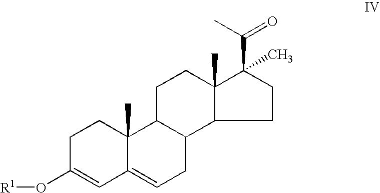
wherein R¹ represents C₁₋₄ alkyl, with dimethyl formamide and phosphorus oxychloride in an organic solvent or solvent mixture which is inert under the reaction conditions.
